(19) United States
(12) Patent Application Publication
(10) Pub. No.: US 2023/0406931 A1
(43) Pub. Date: Dec. 21, 2023

(54) Adjuvant Durvalumab in Combination with Chemotherapy for Treatment of Cancer

(71) Applicant: ASTRAZENECA AB, SODERTALJE (SE)

(72) Inventors: Phillip DENNIS, Gaithersburg, MD (US); Lynne POOLE, Cambridge Cambridgeshire (GB); Rena MAY, Wilmington, DE (US)

(21) Appl. No.: 18/248,486

(22) PCT Filed: Oct. 11, 2021
(86) PCT No.: PCT/EP2021/078050

§ 371 Date: Apr. 10, 2023

Related U.S. Application Data
(60) Provisional application No. 63/090,441, filed on Oct. 12, 2020.

(51) Int. Cl. C07K 16/28; A61K 33/243; A61K 31/519; A61K 31/555; A61K 31/337

(52) U.S. Cl. CPC C07K 16/2818; A61K 33/243; A61K 2039/505; A61K 31/555; A61K 31/337; A61K 31/519

(57) ABSTRACT

The disclosure generally relates to methods for treating cancer in a patient using durvalumab in combination with chemotherapy based on the patient's minimal residual status. Specifically, the disclosure relates to preventing or treating a recurrent tumor in a patient, wherein the patient is minimal residual disease-positive (MRD+), using durvalumab and chemotherapy.

### FIELD OF THE DISCLOSURE

The disclosure generally relates to methods for treating cancer in a patient using durvalumab in combination with chemotherapy based on the patient's minimal residual disease status. Specifically, the disclosure relates to preventing or treating a recurrent tumor in a patient, wherein the patient is minimal residual disease-positive (MRD⁺), using durvalumab and chemotherapy.

### BACKGROUND OF THE DISCLOSURE

As many as 30% of patients with non-small cell lung cancer (NSCLC) present with surgically resectable disease (Molina et al., Mayo Clin. Proc. 83(5): 584-94 (2008)). For patients with stage II-IIIA and select IIIB disease, surgery and adjuvant standard of Care (SoC) chemotherapy results in 5-year disease-free survival (DFS) rates of only ˜40% (Wakelee et al., Lancet. Oncol. 18(12): 1610-23 (2017)). Adjuvant chemotherapy following resection of NSCLC is standard practice to reduce risk of disease recurrence. The majority of patients who remain event-free at 5 years are cured by surgery alone yet receive adjuvant treatment because there is currently no clear way to determine who will benefit from adjuvant chemotherapy.

There is evidence that identification of the minimal residual disease (MRD) status of a patient through detection of circulating tumor DNA (ctDNA) post-surgery can accurately predict disease recurrence (Abbosh et al., Nature 545(7655): 446-51 (2017); Chaudhuri et al., Cancer Discov. 7(12): 1394-403 (2017)). In the TRACERx study, MRD was evaluated via ctDNA detection in plasma samples collected from patients who had undergone surgery for stage III NSCLC. In 13 of 14 patients who underwent surgical resection of their tumors and subsequently suffered postoperative relapse of their disease, MRD was detected via ctDNA before or at the point of clinically evident disease recurrence (through SoC imaging or presentation of symptoms) (Abbosh et al. (2017)). In all 12 patients who did not experience postoperative disease recurrence, MRD was not detected following surgery (Abbosh et al. (2017)). Detection of MRD at a time when there is no radiologic evidence of disease provides an opportunity for earlier therapeutic intervention (Abbosh et al., Nat. Rev. Clin. Oncol. 15(9): 577-86 (2018)). Patients with MRD (MRD-positive (MRD+)) experience inferior recurrence-free survival compared to patients without detectable MRD (MRD-negative (MRD−)). Interestingly, NSCLC patients who were found to be MRD+ after completing chemoradiation therapy had better outcomes if they went on to receive consolidation immunotherapy treatment compared to those MRD+ patients who did not. These data suggest that immunotherapy could improve outcomes for NSCLC patients who are MRD+ after completion of SoC (Moding, et al. Nat Cancer 1, 176-183 (2020).

Durvalumab can be effective in situations of residual cancer as evidenced by improved progression-free survival (PFS) and overall survival (OS) observed with durvalumab versus placebo following definitive concurrent chemoradiation in the PACIFIC study (Antonia et al 2018, Gray et al 2019). Moreover, intervention with combination chemotherapy and immunotherapy versus chemotherapy alone improves PFS and OS in advanced NSCLC (Gandhi et al 2018, Gadgeel et al 2019, Paz-Ares et al 2018). These data suggest that earlier intervention with immunotherapy as adjuvant therapy following curative intent treatment could improve outcomes in early-stage NSCLC, prevent progression, and circumvent the need to expose patients to potentially more toxic chemotherapy regimens in the metastatic setting.

This study described herein shows that detection of MRD via ctDNA isolation after complete resection±neoadjuvant and/or adjuvant therapy for stage II-III NSCLC identifies a high risk patient population that would receive benefit from additional adjuvant therapy. In addition, this study discloses that adjuvant durvalumab monotherapy is more effective than placebo in treating this patient population.

### SUMMARY OF THE DISCLOSURE

The disclosure provides a method of preventing a recurrent tumor in a patient in need thereof, comprising administering durvalumab and chemotherapy to the patient, wherein the patient is minimal residual disease-positive (MRD+).

The disclosure further provides a method of treating a recurrent tumor in a patient in need thereof, comprising: (a) determining whether the patient is minimal residual disease-positive (MRD+); and (b) treating or continuing treatment if the patient is identified as MRD+, wherein the treatment comprises treatment with durvalumab and chemotherapy.

Specific embodiments of the claimed invention will become evident from the following more detailed description of certain embodiments and the claims.

### BRIEF DESCRIPTION OF THE DRAWINGS/FIGURES

FIG. 1 illustrates a schema for the general study design.

FIGS. 2A-2B show the full dosing scheme by treatment arm throughout the 12-month treatment period. FIG. 2A shows the dosing scheme for administration every three weeks for 4 cycles. FIG. 2B shows the dosing scheme for administration every four weeks for 10 cycles.

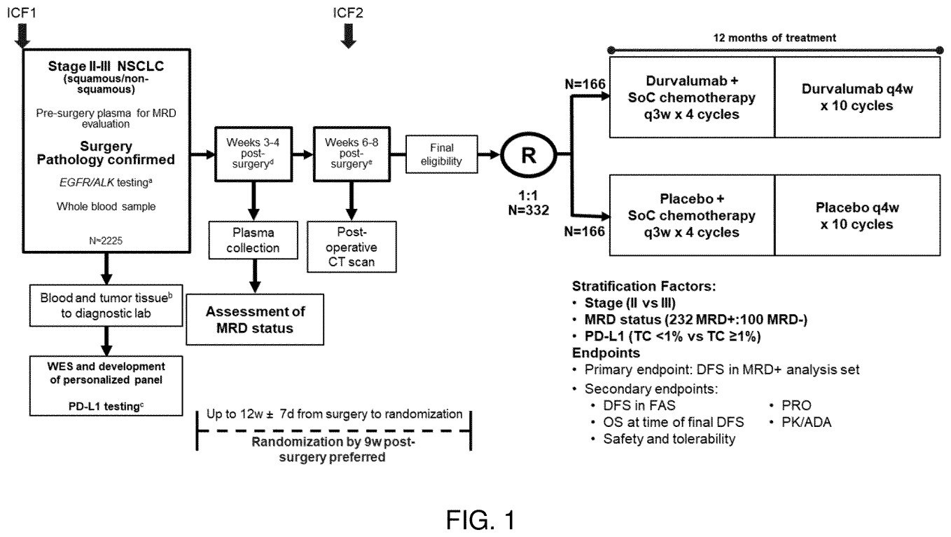

### DETAILED DESCRIPTION OF THE DISCLOSURE

The disclosure generally relates to methods for treating cancer in a patient using durvalumab in combination with chemotherapy based on the patient's minimal residual disease status. Specifically, the disclosure relates to preventing or treating a recurrent tumor in a patient, wherein the patient is minimal residual disease-positive (MRD+), using durvalumab and chemotherapy.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

In particular embodiments provided herein is a method of preventing a recurrent tumor in a patient in need thereof, comprising administering durvalumab and chemotherapy to the patient, wherein the patient is minimal residual disease-positive (MRD+).

In a particular embodiment provided herein is a method of treating a recurrent tumor in a patient in need thereof, comprising: (a) determining whether the patient is minimal residual disease-positive (MRD+); and (b) treating or continuing treatment if the patient is identified as MRD+, wherein the treatment comprises treatment with durvalumab and chemotherapy.

The MRD status of a patient can be determined using methods known in the art (see, e.g., Abbosh et al. (2017); Chaudhuri et al. (2017)). In some embodiments, a patient's MRD status can be determined using a multi-step assay. First, whole exome sequencing (WES) is performed on DNA extracted from the patient's tumor tissue and controlled for germline mutations by WES of the patient's whole blood. A personalized panel is then developed, comprised of the patient's tumor variants expressed at high frequency. This panel is then used to identify the presence of these variants on ctDNA extracted from the patient's plasma and the patient is considered MRD+if the panel detects a tumor variant. This personalized approach allows detection of the patient's tumor variants in DNA extracted from their plasma at high sensitivity.

In some embodiments, determining whether the patient is minimal residual disease-positive (MRD+) is determined by: (a) sequencing all or part of the genome or exome of a tumor of the patient to define clonal and/or subclonal mutations in the tumor; (b) defining a set of reagents that will detect the presence of DNA from the tumor via the presence of the clonal and/or subclonal mutations; and (c) analyzing a sample comprising DNA from the tumor obtained from the patient subsequent to the tumor removal and the defined set of reagents to determine whether the tumor has recurred by detection of the clonal and/or subclonal mutations in the sample. The presence and/or rise of clonal and/or subclonal mutations in the sample from the patient characteristic of the tumor indicates whether the tumor has recurred. The clonal and/or subclonal mutations characteristic of the patient's tumor are defined by sequencing all or part of the whole genome and/or exome of DNA from the tumor, in certain instances after the tumor has been resected from the patient. Using a set of reagents designed or defined to detect the presence of DNA from the tumor via the presence of the specific clonal and/or subclonal mutations identified for the specific subject of interest, the presence and/or rise of clonal and/or subclonal mutations in the sample obtained from the patient is analyzed.

In some embodiments, the sequencing is carried out on a tumor biopsy, all or part of the tumor or one or more subsections of the tumor, cell free DNA (cfDNA), circulating tumor DNA, exosome derived tumor DNA, or circulating tumor cells from the subject. In some embodiments, the sequencing is carried out on the tumor or subsection thereof following removal of the tumor. In some embodiments, all or part of the genome or exome of at least two subsections of the tumor is sequenced and clonal and/or subclonal mutations are defined based on which mutations occur in which tumor subsections. In some embodiments, the defined set of reagents comprise multiplex PCR primers and the analysis is a multiplex PCR. In some embodiments, sequencing is carried out on blood plasma obtained from the patient, or the sample to be analyzed is a blood plasma sample from the patient.

MRD, as indicated by detection of ctDNA, may reveal the existence of clinically indiscernible residual tumor following curative intent therapy (surgery±chemotherapy/radiotherapy). Detection of MRD at a time when there is no radiologic evidence of disease provides an opportunity for earlier therapeutic intervention. MRD+ patients experience inferior recurrence-free survival compared to MRD− patients. Therefore, MRD+ patients may benefit from earlier intervention and escalation of treatment, including immunotherapy alone or in combination with chemotherapy; furthermore, MRD− patients (the majority of whom are cured by surgery alone) could be spared from more intensive therapy and the resulting unnecessary toxicity. Establishing ctDNA clearance as a novel surrogate of overall survival (OS) or disease free survival (DFS) allows for acceleration of the adoption of novel therapeutic strategies in the adjuvant NSCLC setting to clear therapeutically vulnerable residual disease following SoC curative intent therapy.

The present disclosure advantageously provides an advanced, sensitive, personalized assay predicated on sequencing the excised primary tumor alongside a whole blood sample to derive a patient-specific MRD signature. This leads to optimal capture of MRD+ patients prior to adjuvant SoC therapy.

The term “patient” is intended to include human and non-human animals, particularly mammals.

In some embodiments, the methods disclosed herein relate to treating a patient for a tumor disorder and/or a cancer disorder. In some embodiments, the tumor is a lung tumor (e.g., non-small cell lung cancer (NSCLC)), a breast tumor, a colorectal tumor, or a prostate tumor. In some embodiments, the non-small cell lung tumor is a squamous cell carcinoma, an adenocarcinoma, or a large cell carcinoma.

In some embodiments, the patient previously underwent complete resection of a non-small cell lung tumor. In some embodiments, the non-small cell lung tumor was stage I, stage II, or stage III. Adjuvant chemotherapy following resection of NSCLC is standard practice to reduce risk of disease recurrence. In particular embodiments, the treatment is an adjuvant treatment.

The terms “treatment” or “treat,” as used herein, refer to therapeutic treatment. Those in need of treatment include subjects having cancer. In some embodiments, the methods disclosed herein can be used to treat tumors. In other embodiments, treatment of a tumor includes inhibiting tumor growth, promoting tumor reduction, or both inhibiting tumor growth and promoting tumor reduction.

The terms “administration” or “administering,” as used herein, refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, or implants.

Despite improvements in outcome with single agent immune checkpoint inhibition, only a minority of NSCLC patients respond to treatment. Combining chemotherapy with immunotherapy has been demonstrated to improve on these response rates and facilitates more aggressive treatment of patients with metastatic disease prior to decline in performance status that inexorably occurs with progression of disease (PD). Combination of chemotherapy and checkpoint inhibition has also shown benefit as a first-line treatment for treatment-naïve metastatic squamous NSCLC. In particular embodiments, provided herein are methods of treating a patient using a combination treatment comprising durvalumab and chemotherapy. The goal of combination chemotherapy is to utilize agents that affect cancer cells by different mechanisms, thus reducing the risk of developing resistance.

It is increasingly understood that cancers are recognized by the immune system, and under some circumstances, the immune system may control or even eliminate tumors. PD-L1 is part of a complex system of receptors and ligands that are involved in controlling T-cell activation. When PD-L1 binds to PD-1, an inhibitory signal is transmitted into the T cell, which reduces cytokine production and suppresses T-cell proliferation. Tumor cells exploit this immune checkpoint pathway as a mechanism to evade detection and inhibit immune response. PD-L1 is expressed in a broad range of cancers. Nonclinical data have now been added to a wealth of clinical data showing that blockade of negative regulatory signals to T cells such as PD-L1 has promising clinical activity.

In particular embodiments, the method of treatment disclosed herein comprises durvalumab. The term “durvalumab,” as used herein, refers to an antibody that selectively binds PD-L1 and blocks the binding of PD-L1 to the PD-1 and CD80 receptors, as disclosed in U.S. Pat. No. 9,493,565 (wherein durvalumab is referred to as “2.14H9OPT”), which is incorporated by reference herein in its entirety. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (“ADCC”). Durvalumab can relieve PD-L1-mediated suppression of human T-cell activation in vitro and inhibits tumor growth in a xenograft model via a T-cell dependent mechanism.

In some embodiments, the chemotherapy comprises a platinum-based chemotherapy agent.

In some embodiments, the chemotherapy comprises at least one of paclitaxel, carboplatin, pemetrexed, or cisplatin.

The dose of durvalumab and the chemotherapy to be administered to the patient will vary depending, in part, upon the size (body weight, body surface, or organ size) and condition (the age and general health) of the patient.

In particular embodiments, durvalumab and chemotherapy are administered over a two-week treatment period, over a four-week treatment period, over a six-week treatment period, over an eight-week treatment period, over a twelve-week treatment period, over a twenty-four-week treatment period, or over a one-year or more treatment period. In particular embodiments, durvalumab and the chemotherapy are administered over a three-week treatment period, over a six-week treatment period, over a nine-week treatment period, over a twelve-week treatment period, over a twenty-four-week treatment period, or over a one-year or more treatment period. In particular embodiments, durvalumab and the chemotherapy are administered over a two-month treatment period, over a four-month treatment period, over a six-month treatment period, or over a twelve-month treatment period.

In particular embodiments, durvalumab and the chemotherapy are administered every two weeks, every two weeks, every four weeks, every five weeks, every six weeks, every seven weeks, every eight weeks, every nine weeks, or every ten weeks.

In particular embodiments, durvalumab and the chemotherapy are administered every three weeks for four doses followed by administration of durvalumab every four weeks.

In particular embodiments, durvalumab and the least one chemotherapy agent are administered simultaneously, concurrently, separately, or sequentially. In some embodiments, durvalumab is administered prior to the chemotherapy. In further embodiments, durvalumab is administered concurrently with chemotherapy.

In particular embodiments, the method further comprises administration of radiotherapy to the patient. For example, patients with pathologically confirmed N2 disease or positive pleural margins will receive adjuvant postoperative radiation therapy (PORT), provided that radiation therapy is given sequential to chemotherapy (i.e., during durvalumab or placebo monotherapy) but not concurrent to chemotherapy. In particular embodiments, the patient is administered a dose ranging from 50 to 60 Gy, 1.8 to 2 Gy per fraction, or 5 fractions a week. In particular embodiments, the radiotherapy is intensity-modulated radiation therapy (IMRT) or 3D-conformal radiotherapy.

In some embodiments, the success of a treatment is determined by an increase in disease free survival (DFS) as compared to standard of care. DFS is defined as the time from the date of randomization until any one of the following events:
Disease recurrence
   Local, regional, or distant disease recurrence,
Diagnosis of a second primary NSCLC,
   OR
Death from any cause
   “Standard of care” (SoC) and “platinum-based chemotherapy” refer to chemotherapy treatment comprising at least one of paclitaxel, carboplatin, pemetrexed, or cisplatin.

When used for in vivo administration, the formulations of the disclosure should be sterile. The formulations of the disclosure may be sterilized by various sterilization methods, including, for example, sterile filtration, or radiation. In one embodiment, the formulation is filter sterilized with a presterilized 0.22-micron filter. Sterile compositions for injection can be formulated according to conventional pharmaceutical practice as described in “Remington: The Science & Practice of Pharmacy,” 21st ed., Lippincott Williams & Wilkins (2005).

The formulations can be presented in unit dosage form and can be prepared by any method known in the art of pharmacy. Actual dosage levels of the active ingredients in the formulation of the present disclosure may be varied so as to obtain an amount of the active ingredient which is effective to achieve the desired therapeutic response for a particular subject, composition, and mode of administration, without being toxic to the subject (e.g., “a therapeutically effective amount”). Dosages can also be administered via continuous infusion (such as through a pump). The administered dose may also depend on the route of administration. For example, subcutaneous administration may require a higher dosage than intravenous administration.

### EXAMPLES

The Examples that follow are illustrative of specific embodiments of the disclosure, and various uses thereof. They are set forth for explanatory purposes only, and should not be construed as limiting the scope of the claimed invention in any way.

### Example 1: Efficacy of Adjuvant Durvalumab in Combination with Platinum-Based Chemotherapy in Completely Resected Stage II-III NSCLC

Disclosed herein is a Phase III, randomized, multicenter, double-blind, placebo-controlled study to evaluate the efficacy and safety of durvalumab adjuvant therapy compared to placebo in patients with stage II-III NSCLC who have undergone curative intent therapy (complete resection±neoadjuvant and/or adjuvant therapy), who have no evidence of RECIST 1.1-defined disease recurrence, and who become MRD+ during a 96-week surveillance period. The general study design is summarized in Error! Reference source not found. The primary objective of the study is to assess the efficacy of durvalumab+SoC chemotherapy as compared to placebo+SoC chemotherapy as measured by disease-free survival (DFS) in minimal residual disease positive (MRD+) patients. Historical data have shown that the DFS benefit seen in MRD+ patients treated with adjuvant chemotherapy was consistent with an improvement in the OS outcome, which suggests an association between these two endpoints in this setting (Mauguen et al., Lancet Oncol. 14(7): 619-26 (2013)). Additional objectives are to assess the efficacy of durvalumab+SoC chemotherapy to clear ctDNA in MRD+ patients as compared to placebo+SoC chemotherapy; to assess the relationship between treatment effect on DFS and treatment effect on ctDNA endpoints; to assess prognostic significance of MRD detection as determined by ctDNA in NSCLC; to assess the association of tumor mutational burden (TMB) with efficacy of durvalumab+SoC chemotherapy as compared with placebo+SoC chemotherapy; and to investigate the relationship between a patient's baseline PD-L1 tumor cell (TC) expression and efficacy outcomes with durvalumab+SoC chemotherapy as compared with placebo+SoC chemotherapy. The SoC options provided in the study include agents that are commonly used in adjuvant therapy. Table 1 shows the study treatments used in the study.

TABLE 1

| | | | Standard of Care | | |
|---|---|---|---|---|---|
| | | | Squamous | | |
| Study | | | histology | Non-squamous histology | |
| treatment | | | Carboplatin/ | Cisplatin | Carboplatin |
| name: | Durvalumab | Placebo | Paclitaxel | Pemetrexed | Pemetrexed |
| Dosage | 500-mg vial | Vial solution | As sourced | As sourced | As sourced |
| formulation: | solution for | for infusion | locally | locally | locally |
| | infusion after | after dilution | | | |
| | dilution, | | | | |
| | 50 mg/mL | | | | |
| Route of | IV | IV | IV | IV | IV |
| administration | | | | | |
| Dosing | 1500 mg | Infusion over | Paclitaxel | Pemetrexed | Pemetrexed |
| instructions: | infusion over | 60 min q3w × | (200 mg/m²) | (500 mg/m²) | (500 mg/m²) |
| | 60 min q3w × | 4 cycles | and carboplatin | and cisplatin | with |
| | 4 cycles | followed by | (AUC 6) | (75 mg/m²) | carboplatin |
| | followed by | infusion over | Day 1 of each | Day 1 of | (AUC 5) |
| | 1500 mg over | 60 min q4w × | 3-week cycle | each 3-week | Day 1 of |
| | 60 min q4w × | 10 cycles up | for 4 cycles | cycle for | each 3-week |
| | 10 cycles up | to a total of 12 | | 4 cycles | cycle for |
| | to a total of 12 | months of | | | 4 cycles |
| | months of | treatment | | | |
| | treatment | | | | |
| Packaging | Study | Study | Sourced | Sourced | Sourced |
| and labeling | treatment will | treatment will | locally by | locally by | locally by |
| | be provided in | be provided in | site | site | site |
| | 500 mg vials. | vials. Each | | | |
| | Each 500 mg | vial will be | | | |
| | vial will be | labeled in | | | |
| | labeled in | accordance | | | |
| | accordance | with Good | | | |
| | with Good | Manufacturing | | | |
| | Manufacturing | Practice | | | |
| | Practice | (GMP) Annex | | | |
| | (GMP) Annex | 13 and per | | | |
| | 13 and per | country | | | |
| | country | regulatory | | | |
| | regulatory | requirement. | | | |
| | requirement. | | | | |
| Provider | AstraZeneca | AstraZeneca | Sourced | Sourced | Sourced |
| | | | locally by | locally by | locally by |
| | | | site | site | site |

The ctDNA endpoints for the study are defined by:
Best overall clearance rate (number converted at any time)
Best confirmed clearance rate (as above but confirmed at subsequent visit)
Time to ctDNA clearance
Duration of ctDNA clearance
Time to ctDNA recurrence
Time to confirmed ctDNA recurrence
Changes in variant allele frequencies (VAF) following treatment

Efficacy assessments of the primary endpoint of DFS will be derived according to RECIST 1.1 guidelines and prespecified definitions of disease recurrence (i.e., local or regional recurrence, distant recurrence, second primary NSCLC) and by survival assessments. All patients will be followed for disease recurrence until the primary analysis, and followed for survival until the completion of the study. DFS will be analyzed using a stratified log-rank test. The treatment effect will be estimated in terms of hazard ratio (HR) together with the corresponding 95% confidence interval (CI) from a Cox proportional hazard model stratified by disease stage, PD-L1 status, and MRD status. For the primary analysis in the MRD+ analysis set, the MRD status stratification factor will not be included. Subgroup analyses will be conducted in the following subgroups (but not limited to these subgroups) comparing DFS between durvalumab plus SoC chemotherapy versus placebo plus SoC chemotherapy in both the MRD+ analysis set and full analysis set:
PD-L1 status (TC<1% versus ≥1%)
PD-L1 status (TC<25% and ≥25%)
TMB (high, low)

This study uses a 2-tiered informed consent and screening process such that initial inclusion criteria are assessed during the first screening period and additional inclusion/exclusion criteria are assessed during the second screening period. The study will screen approximately 1500-2300 patients and randomize approximately 230-340 MRD+ patients with stage II-III NSCLC (according to IASLC Staging Manual in Thoracic Oncology v8.0) whose tumors are EGFR and ALK wildtype, and who have completed curative intent therapy.

This study also requires mandatory genetic testing. During the first screening, whole exome sequencing (WES) is performed on the patient's resected tumor tissue and derived tumor-specific DNA variants are identified by removing background germline variants, determined by WES of the patient's whole blood sample. A personalized panel is then created, comprised of up to 50 of the patient's tumor variants present at a high frequency. This panel is then used to identify the presence of these tumor-specific variants on DNA extracted from the patient's plasma. The patient is considered MRD+ if the panel detects patient-specific tumor variants.

Resected tumor tissue collected during the first screening will be evaluated for EGFR/ALK and programmed death ligand-1 (PD-L1) expression by a central reference laboratory. Patients whose tumor tissue tests positive for EGFR mutations and/or ALK translocations will be excluded from the study. In addition, PD-L1 status must be known prior to, and is required for, randomization.

Eligible patients will be enrolled in a 96-week surveillance period during which they will be monitored for the emergence of MRD. During surveillance, the patient will be assessed for MRD by plasma sampling every 6 weeks (q6w±3d) and will receive CT scans every 12 weeks (q12w±1w) for up to 96 weeks. Patients with evidence of RECIST 1.1-defined disease recurrence during the surveillance period will not be eligible for randomization and will no longer be followed as part of the study; however, data pertaining to their recurrence must be captured. Patients who become MRD+ during surveillance (including cases where analysis of the first plasma sample collected [marking the start of surveillance] returns an MRD+ status) will undergo a second screening period. Patients who received prior neoadjuvant immunotherapy must be MRD− based on analysis of the first plasma sample collected (which marks the start of surveillance).

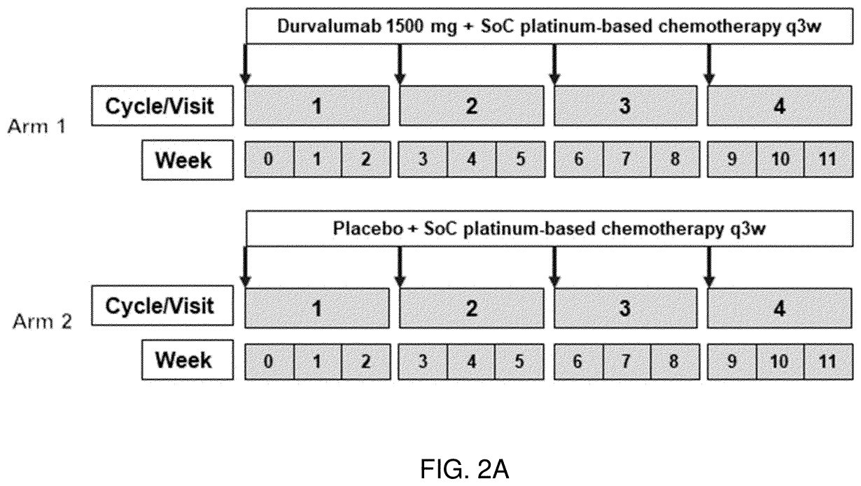

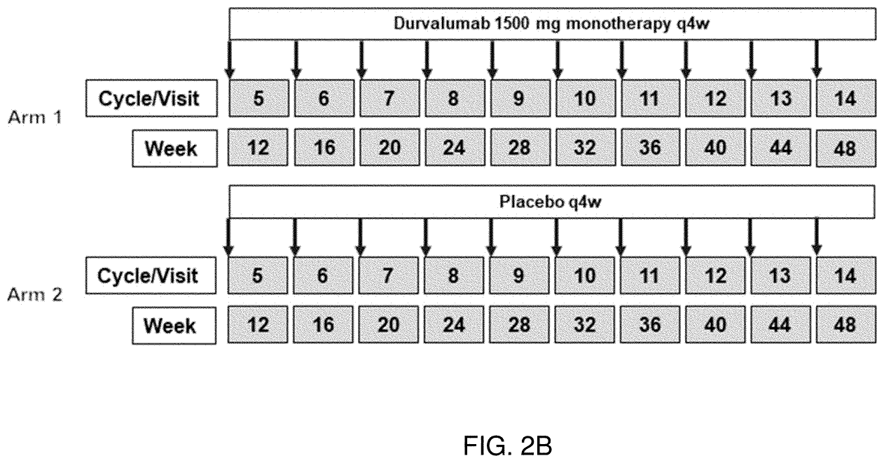

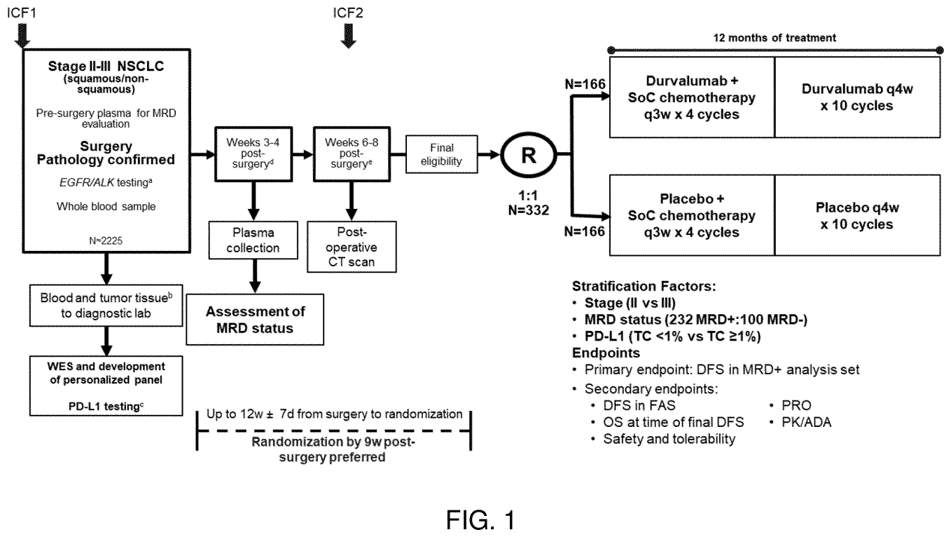

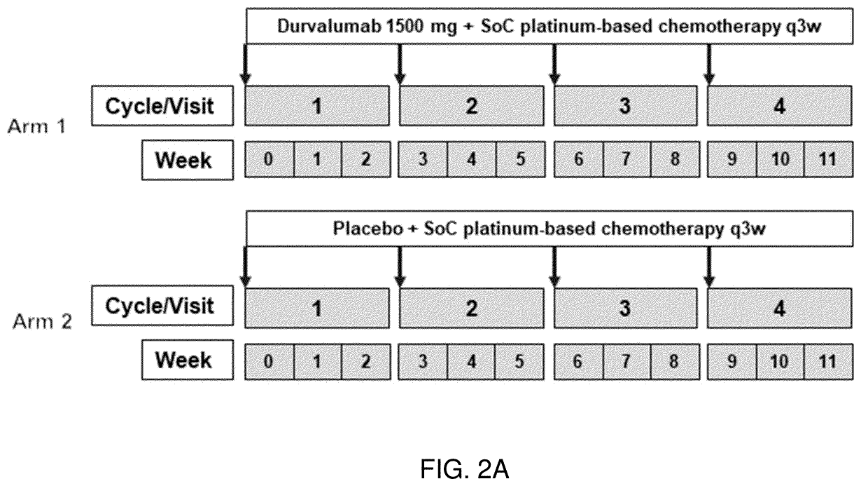

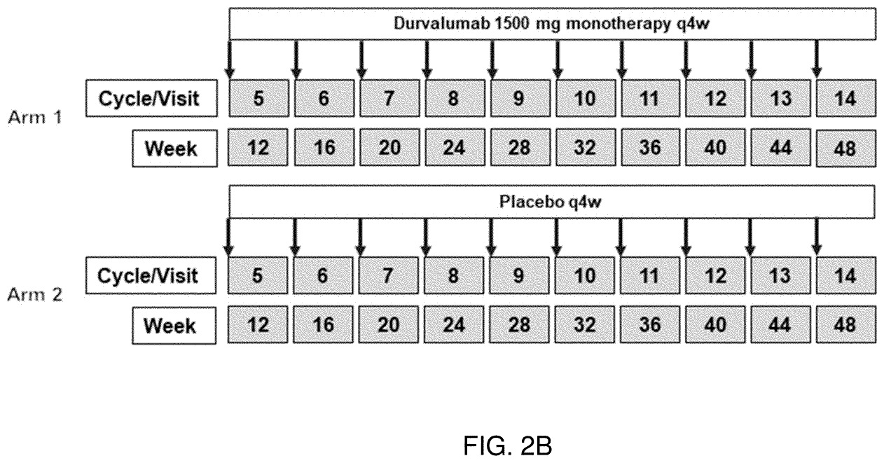

Once all additional inclusion and none of the exclusion criteria are met, patients will be eligible for randomization. Patients must be randomized as soon as possible after eligibility criteria are confirmed, and treatment must begin within 3 days of randomization. Approximately 230-290 MRD+ patients will be randomized 1:1 to one of two treatment regiments (FIG. 2):
(1) treatment with durvalumab monotherapy 1500 mg or placebo every 4 weeks (q4w) intravenously (IV). Patients will be treated for up to a total of 24 months (26 cycles), until disease recurrence, or until other specific treatment discontinuation criteria are met (whichever comes first); or
(2) treatment with durvalumab every 3 weeks (q3w) for 4 cycles plus SoC chemotherapy q3w for up to 4 cycles or placebo q3w for 4 cycles plus SoC chemotherapy q3w for up to 4 cycles followed by durvalumab or placebo every four weeks (q4w) for up to an additional 10 cycles (for a total of 12 months of treatment), until disease recurrence, or until other specific treatment discontinuation criteria are met (whichever occurs first).
   It is estimated that 60% of randomized patients will be PD-L1 TC≥1%. To ensure that the target of 170 PD L1 TC≥1% patients are randomized in the study, enrollment of PD-L1 TC<1% patients into surveillance will end once 114 PD-L1 TC<1% patients have been randomized. The remaining PD-L1 TC<1% patients will be immediately withdrawn from surveillance. Additional data will not be collected on these patients. Patients who are determined to be PD-L1 TC<1% during the first screening will not be eligible to continue to surveillance and will be considered screen failures. Any PD-L1 TC<1% patient who is already in the observation arm will remain in the study. Similarly, enrollment into the study will end once 284 MRD+ patients are randomized. Patients in surveillance will be immediately withdrawn from the study and additional data will not be collected on these patients. Patients already in the observation arm will remain in the study.

Up to 142 of the patients who complete their 96-week surveillance period, remain MRD−, and have no evidence of RECIST 1.1-defined disease recurrence may be eligible for entry into an observation period. These patients will be followed for SAEs, DFS, OS, subsequent anticancer therapy, and MRD assessments for 24 months or until completion of the study (whichever occurs first). Patients who complete 24 months of observation will be followed for OS until the end of the study. Data from this cohort of patients will be compared to the patients randomized to placebo to support the conclusion that MRD can be used as a prognostic biomarker to identify patients at high risk of disease recurrence prior to radiologic evidence of disease. After completion of durvalumab or placebo treatment, patients will be followed for safety, ctDNA, disease recurrence, and survival status at specified intervals until the primary DFS analysis. Patients will be followed for long-term survival until the end of the study.

All patents and publications mentioned in this specification are herein incorporated by reference to the same extent as if each independent patent and publication was specifically and individually indicated to be incorporated by reference. Citation or identification of any reference in any section of this application shall not be construed as an admission that such reference is available as prior art to the claimed invention.

1: A method of preventing a recurrent tumor in a patient in need thereof, comprising administering durvalumab and chemotherapy to the patient, wherein the patient is minimal residual disease-positive (MRD+).

2: The method of claim 1, wherein the chemotherapy comprises at least one of paclitaxel, carboplatin, pemetrexed, or cisplatin.

3: The method of claim 1, wherein the tumor is a lung tumor, a breast tumor, a colorectal tumor, a prostate tumor, or a melanoma.

4: The method of claim 3, wherein the tumor is a non-small cell lung tumor.

5: The method of claim 4, wherein the non-small cell lung tumor is a squamous cell carcinoma, an adenocarcinoma, or a large cell carcinoma.

6: The method of claim 4, wherein the patient previously underwent complete resection of a non-small cell lung tumor.

7: The method of claim 6, wherein the non-small cell lung tumor was stage I, stage II, or stage III.

8: The method of claim 1, wherein durvalumab and the chemotherapy are administered every three weeks.

9: The method of claim 1, wherein durvalumab and the chemotherapy are administered every four weeks.

10: The method of claim 1, wherein durvalumab and the chemotherapy are administered every three weeks for four doses followed by administration of durvalumab every four weeks.

11: The method of claim 1, wherein durvalumab and the least one chemotherapy are administered simultaneously, separately, or sequentially.

12: The method of claim 1, wherein the administration of durvalumab and the chemotherapy results in increased disease free survival as compared to standard of care.

13: A method of treating a recurrent tumor in a patient in need thereof, comprising:
(a) determining whether the patient is minimal residual disease-positive (MRD+); and
(b) treating or continuing treatment if the patient is identified as MRD+, wherein the treatment comprises treatment with durvalumab and chemotherapy.

14: The method of claim 13, wherein the chemotherapy comprises at least one of paclitaxel, carboplatin, pemetrexed, or cisplatin.

15: The method of claim 13, wherein the tumor is a lung tumor, a breast tumor, a colorectal tumor, a prostate tumor, or a melanoma.

16: The method of claim 15, wherein the tumor is a non-small cell lung tumor.

17: The method of claim 16, wherein the non-small cell lung tumor is a squamous cell carcinoma, an adenocarcinoma, or a large cell carcinoma.

18: The method of claim 16, wherein the patient previously underwent complete resection of a non-small cell lung tumor.

19: The method of claim 18, wherein the non-small cell lung tumor was stage I, stage II, or stage III.

20: The method of claim 13, wherein durvalumab and the chemotherapy are administered every three weeks.

21: The method of claim 13, wherein durvalumab and the chemotherapy are administered every four weeks.

22: The method of claim 13, wherein durvalumab and the chemotherapy are administered every three weeks for four doses followed by administration of durvalumab every four weeks.

23: The method of claim 13, wherein durvalumab and the least one chemotherapy are administered simultaneously, separately, or sequentially.

24: The method of claim 13, wherein whether the patient is minimal residual disease-positive (MRD+) is determined by:
(a) sequencing all or part of the genome or exome of a tumor of the patient to define clonal and/or subclonal mutations in the tumor;
(b) defining a set of reagents that will detect the presence of DNA from the tumor via the presence of the clonal and/or subclonal mutations; and
(c) analyzing a sample comprising DNA from the tumor obtained from the patient subsequent to the tumor removal and the defined set of reagents to determine whether the tumor has recurred by detection of the clonal and/or subclonal mutations in the sample.

25: The method of claim 24, wherein the sequencing is carried out on a tumor biopsy, all or part of the tumor or one or more subsections of the tumor, cell free DNA (cfDNA), circulating tumor DNA, exosome derived tumor DNA, or circulating tumor cells from the subject.

26: The method of claim 25, wherein the sequencing is carried out on the tumor or subsection thereof following removal of the tumor.

27: The method of claim 24, wherein all or part of the genome or exome of at least two subsections of the tumor is sequenced and clonal and/or subclonal mutations are defined based on which mutations occur in which tumor subsections.

28: The method of claim 24, wherein the defined set of reagents comprise multiplex PCR primers and the analysis is a multiplex PCR.

29: The method of claim 24, wherein the sequencing is carried out on blood plasma obtained from the patient, or the sample to be analyzed is a blood plasma sample from the patient.

30: The method of claim 13, wherein the treatment is an adjuvant treatment.

31: The method of claim 13 further comprising administration of radiotherapy.

32: The method of claim 13, wherein the administration of durvalumab and the chemotherapy results in increased disease free survival as compared to standard of care.
